(19) United States
(12) Patent Application Publication
(10) Pub. No.: US 2018/0043080 A1
(43) Pub. Date: Feb. 15, 2018

(54) CATHETER AND PERITONEUM HEALTH MONITORING

(71) Applicant: Medtronic, Inc., Minneapolis, MN (US)

(72) Inventors: Martin T. GERBER, Maple Grove, MN (US); Christopher M. HOBOT, Rogers, MN (US)

(21) Appl. No.: 15/673,487

(22) Filed: Aug. 10, 2017

Related U.S. Application Data
(60) Provisional application No. 62/373,225, filed on Aug. 10, 2016.

(51) Int. Cl. A61M 1/28; A61B 5/03; A61B 5/145

(52) U.S. Cl. CPC A61M 1/282; A61B 5/14532; A61B 5/14539; A61B 5/03; A61B 5/14507; A61M 2205/3344; A61M 2205/3317; A61M 2205/3324; A61M 2205/3303; A61M 2205/3334; A61M 2205/3368; A61M 2205/3306; A61M 2205/52; A61M 2205/18

(57) ABSTRACT

The invention relates to systems and methods for monitoring the long term health of the peritoneum and catheter in a patient undergoing peritoneal dialysis (PD) treatment. The systems and methods include processors and sensors for determining changes in the peritoneum health or catheter of a PD patient to support analysis, replacement, and possible medical intervention. The method can include receiving a prior history of a patient; storing the prior history in a machine-readable storage medium, which when executed performs the steps of trending the one or more fluid parameters from the prior history; and determining a change in peritoneum health or catheter patency from the trended prior history. The system can include a peritoneal dialysis cycler having an infusion line and an effluent line; at least one sensor positioned in the infusion line, the effluent line, or combinations thereof; and a processor in communication with the sensor.

### CROSS-REFERENCE TO RELATED APPLICATIONS

This application claims the benefit of and priority to U.S. Provisional Patent Application No. 62/373,225 filed Aug. 10, 2016, the entire disclosure of which is incorporated by reference herein.

### FIELD OF THE INVENTION

The invention relates to systems and methods for monitoring the long term health of the peritoneum and catheter in a patient undergoing peritoneal dialysis (PD) treatment. The systems and methods include processors and sensors for determining changes in the peritoneum health or catheter of a PD patient to support analysis, replacement, and possible medical intervention.

### BACKGROUND

Over the course of peritoneal dialysis (PD) therapy, patients may experience problems such as infection, peritonitis, a failing peritoneum, or problems with the catheter used to deliver peritoneal dialysis therapy. Current systems and methods do not monitor catheter health and/or performance from one PD session to another. Problems associated with catheters include tissue in-growth which can ultimately occlude the catheter, catheter wear-out, catheter defects, and kinking. Early intervention of any such problems can drastically improve the long-term health of a patient. However, known systems do not provide any mechanism to determine changes in the catheter or peritoneum health of a patient undergoing treatment. The known systems do not provide a history or contain the necessary components required for trend analysis, evaluation, possible intervention, and prediction for replacement. Known systems also do not contain the necessary sensors and monitoring components to tack, monitor, and assess catheter performance to support analysis from session to session.

Hence, there is a need for systems and methods of monitoring the catheter and peritoneum of the patient to detect any problems at the earliest possible time. The need extends to systems and methods monitoring the peritoneum and catheter of a patient using sensors positioned in a peritoneal dialysis cycler. The need includes sensors such as refractive index sensors, pH sensors, conductivity sensors, fluid flow sensors, pressure sensors, temperature sensors, urea sensors, spectroscopes, and other sensors in the peritoneal dialysis cycler. The need includes recording and maintaining a history of catheter and peritoneum monitoring to support trend analysis, evaluation, possible intervention, and prediction for replacement. There is also a need for systems and methods to automatically generate alerts to the patient and health care providers of possible infection, catheter problems, or any other issues affecting the health of the patient.

### SUMMARY OF THE INVENTION

The first aspect of the invention relates to a computer implemented method of monitoring a catheter and/or peritoneum health. In any embodiment, the method can comprise the steps of receiving a prior history of a patient; the prior history of the patient including at least one fluid parameter of the group comprising: pressure to infuse peritoneal dialysate into a patient, flow rate of peritoneal dialysate, ultrafiltrate (UF) transfer efficiency, toxin transfer, protein leakage, fluid flow rate, dialysate effluent optical color and clarity, effluent temperature, intraperitoneal pressure, membrane transfer efficiency, bacteria testing, white cell count, ultrafiltration volume, effluent refractive index, effluent boiling point elevation, effluent freezing point elevation, and effluent urea concentration; storing the prior history in a machine-readable storage medium for storing instructions, which when executed by a processor performs the steps of trending the at least one fluid parameter from the prior history of the patient; and determining a change in peritoneum health or catheter patency from a trended prior history.

In any embodiment, the method can comprise the step of receiving one or more fluid parameters from a peritoneal dialysis session into the machine readable storage medium; comparing the one or more fluid parameters from the peritoneal dialysis session to the trended prior history; and providing an alert if the one or more fluid parameters differ from the trended prior history by a predetermined threshold.

In any embodiment, the fluid parameters from the peritoneal dialysis session can include a fluid flow rate and pressure to infuse peritoneal dialysate into a patient; and the method can include determining a correlation between flow rate and pressure to infuse peritoneal dialysate into a patient from the prior history; and the step of providing an alert can comprise providing a catheter occlusion or catheter kinking alert if the correlation between flow rate and pressure from the peritoneal dialysis session differs from the correlation between flow rate and pressure from the prior history by a predetermined threshold.

In any embodiment, the predetermined threshold can be a pressure increase of more than 25% at a given fluid flow rate.

In any embodiment, the method can comprise the step of providing a fluid bolus to the patient if the correlation between flow rate and pressure from the peritoneal dialysis session differs from the correlation between flow rate and pressure from the prior history by the predetermined threshold.

In any embodiment, the prior history can include the dialysate effluent optical color and clarity; and the method can comprise the step of providing an alert indicating an infection if a trend of dialysate effluent optical color and clarity changes by a predetermined threshold.

In any embodiment, the prior history can include the effluent temperature; and the method can comprise the step of providing an alert indicating an infection if a trend of dialysate effluent temperature changes by a predetermined threshold.

In any embodiment, the prior history can include intraperitoneal pressure; and the method can comprise the step of providing an alert indicating night enteric peritonitis if a trend of intraperitoneal pressure changes by a predetermined threshold.

In any embodiment, the prior history can include protein leakage; and the method can comprise the step of providing an alert indicating membrane wear out if a trend of protein leakage increases by a predetermined threshold.

In any embodiment, the prior history can include membrane transfer efficiency; and the method can comprise the step of providing an alert if a trend of membrane transfer efficiency decreases by a predetermined threshold.

In any embodiment, the method can comprise the steps of receiving a first effluent solute concentration at a first time during a peritoneal dialysis session, receiving a second effluent solute concentration at a second time during the peritoneal dialysis session, and determining the membrane transfer efficiency based on a difference between the first effluent solute concentration and the second effluent solute concentration.

In any embodiment, the method can comprise the step of receiving an ultrafiltration volume, a dwell time, an osmotic agent concentration, and a cycle number from a dialysis cycle; and the membrane transfer efficiency can be determined by the ultrafiltration volume, the dwell time, the osmotic agent concentration, and the cycle number from a dialysis cycle.

In any embodiment, at least one fluid parameter can be received from a sensor in an effluent line of an integrated cycler.

In any embodiment, the prior history of the patient can include at least two fluid parameters.

In any embodiment, each of the at least two fluid parameters can be trended, and the step of determining a change in peritoneum health or catheter patency from the trended prior history can comprise determining a change in peritoneum health or catheter patency from a combination of the trended prior history of the at least two fluid parameters.

In any embodiment, the prior history of the patient can include at least two of: dialysate effluent optical color and clarity, effluent temperature, and effluent pH; and the method can comprise the step of providing an alert indicating an infection if a combined trend of dialysate effluent optical color and clarity, effluent temperature, and effluent pH changes by a predetermined threshold.

In any embodiment, the prior history of the patient can include at least pressure fluctuations and flow fluctuations; and the method can comprise the step of providing an alert indicating a catheter occlusion or catheter kinking if the pressure fluctuations and flow fluctuations exceed a predetermined threshold.

In any embodiment, the prior history of the patient can include at least two of effluent pH, effluent optical clarity, and effluent optical color; and the method can comprise providing an alert indicating peritonitis or an infection if the effluent pH, effluent optical clarity and effluent optical color change by a predetermined threshold.

In any embodiment, the prior history can include at least two of effluent pH, effluent optical clarity, effluent optical color, effluent temperature, bacteria testing, and white cell count; and the method can comprise providing an alert indicating either peritonitis or infection if the effluent pH, effluent optical clarity, effluent optical color, effluent temperature, bacteria testing, and white cell count change by a predetermined threshold.

In any embodiment, the prior history can include at least two of pressure to infuse peritoneal dialysate into a patient, flow rate of peritoneal dialysate, effluent optical color, and effluent optical clarity; and the method can comprise providing an alert indicating catheter occlusion if the pressure to infuse peritoneal dialysate into a patient, flow rate of peritoneal dialysate, effluent optical color, and effluent optical clarity change by a predetermined threshold.

In any embodiment, the prior history can include at least ultrafiltration volume and membrane transfer efficiency; and the method can comprise providing an alert indicating a change in peritoneum health if the ultrafiltration volume and membrane transfer efficiency change by a predetermined threshold.

In any embodiment, the prior history can include a glucose concentration and a dextrose concentration.

In any embodiment, the prior history can include at least two of an effluent refractive index, an effluent boiling point elevation; and an effluent freezing point depression.

In any embodiment, the prior history can include an effluent urea concentration.

The features disclosed as being part of the first aspect of the invention can be in the first aspect of the invention, either alone or in combination.

The second aspect of the invention is drawn to a computer implemented method of monitoring a catheter and/or peritoneum health. In any embodiment, the method can comprise receiving a pressure to infuse peritoneal dialysate into a patient from a peritoneal dialysis cycle; storing the pressure to infuse peritoneal dialysate into the patient in a machine-readable storage medium for storing instructions, which when executed by a processor performs the steps of: providing an alert if the pressure to infuse peritoneal dialysate into the patient exceeds a predetermined threshold.

In any embodiment, the method can comprise the step of providing a fluid bolus to the patient if the pressure to infuse peritoneal dialysate into the patient exceeds a predetermined threshold.

The features disclosed as being part of the second aspect of the invention can be in the second aspect of the invention, either alone or in combination.

The third aspect of the invention is drawn a system. In any embodiment, the system can comprise a peritoneal dialysis cycler comprising an infusion line and an effluent line or a combined effluent and infusion line; at least one sensor positioned in the infusion line, the effluent line, or combinations thereof; and a processor in communication with the at least one sensor; the processor performing the method of the first aspect of the invention.

In any embodiment, the system can comprise a detachable sampling reservoir fluidly connected to the effluent line or combined effluent and infusion line.

In any embodiment, the sensor can be a pressure sensor located in the infusion line or combined effluent and infusion line.

In any embodiment, the sensor can be a conductivity sensor positioned in the effluent line combined effluent and infusion line.

In any embodiment, the sensor can be a pH sensor positioned in the effluent line combined effluent and infusion line.

The features disclosed as being part of the third aspect of the invention can be in the third aspect of the invention, either alone or in combination.

The fourth aspect of the invention is drawn to a sensor suite for monitoring catheter and/or peritoneum health. In any embodiment, the sensor suite can comprise at least one of: (a) a pressure sensor measuring a pressure infusing peritoneal dialysate into a patient; (b) an ultrafiltrate sensor measuring a membrane transfer efficiency across a peritoneal membrane, wherein the ultrafiltrate sensor measures at least one of: (i) toxin transfer; or (ii) protein leakage; (c) a fluid flow sensor; or (d) a dialysate effluent sensor to measure at least one of (i) effluent color; (ii) effluent clarity; or (iii) effluent temperature.

In any embodiment, the sensor suite can comprise at least one of a pH sensor or a conductivity sensor.

In any embodiment, the sensor suite can comprise at least two sensors.

In any embodiment, at least one sensor can be in a peritoneal dialysis cycler.

In any embodiment, the sensor suite can include at least the fluid flow sensor and the pressure sensor.

The features disclosed as being part of the fourth aspect of the invention can be in the fourth aspect of the invention, either alone or in combination,

The fifth aspect of the invention can be drawn to a sensor suite for dialysis performance and/or peritoneum health comprising a group of sensors monitoring at least one of dialysis performance and/or peritoneum health condition selected from the group of inflammation, peritonitis, catheter obstruction for blockage of the catheter, peritoneal membrane health for transport status of the membrane, glucose concentration, total osmolality, and Kt/V for peritoneal dialysis, and/or hemodialysis, wherein the total osmolality is a sum of osmolalities of all solutes present in the peritoneal dialysis and wherein in Kt/V, wherein K stands for dialyzer clearance of urea, t stands for dialysis time, and V stands for a volume of water that the patient's body contains.

In any embodiment, the sensor suite can be selected from the group of a pressure sensor measuring a pressure infusing a peritoneal dialysate into a patient; an ultrafiltrate sensor measuring a membrane transfer efficiency across a peritoneal membrane, wherein the ultrafiltrate sensor measures at least one of: (i) toxin transfer; or (ii) protein leakage; and a fluid flow sensor; or a dialysate effluent sensor measuring at least one of (i) effluent color; (ii) effluent clarity; or (iii) effluent temperature.

In any embodiment, the dialysis performance Kt/V for peritoneal dialysis can measure the conversion of urea to ammonia.

In any embodiment, the dialysis performance Kt/V for peritoneal dialysis can measure the conversion of urease to ammonia combined with the Kt/V measurement of residual kidney function.

In any embodiment, the sensor suite can comprise one or more sensors monitoring dialysis performance via measuring catheter obstruction selected from the group of a flow rate sensor, pressure sensor, and an optical sensor.

In any embodiment the sensor suite can comprise one or more ultrafiltrate sensors monitoring peritoneum health measuring transport across the peritoneal membrane, Urea Reduction Ratio (URR) trending to determine changes in the URR over time, and urea trending to determine changes in urea over time in a peritoneal fluid.

In any embodiment, the sensor suite can comprise one or more sensors monitoring peritoneum health selected from the group of a glucose sensor and a sensor measuring refractive index changes of dextrose in a peritoneal fluid.

In any embodiment, the sensor suite can comprise one or more osmolality sensors monitoring peritoneum health selected from the group of a refractive index sensor, boiling point elevation, and freezing point depression.

In any embodiment, the sensor suite can comprise one or more infection sensors monitoring peritoneum health selected from the group of a pH sensor, a color sensor, a clarity sensor, a temperature sensor, a bacterial concentration test, and a white cell concentration test.

In any embodiment, the sensor suite can comprise one or more peritonitis sensor monitoring peritoneum health selected from the group of a pH sensor, a color sensor, a clarity sensor, a temperature sensor, a bacterial concentration test, and a white cell concentration test.

The features disclosed as being part of the fifth aspect of the invention can be in the fifth aspect of the invention, either alone or in combination.

### BRIEF DESCRIPTION OF THE DRAWINGS

FIG. 1 shows a flow chart of a method of monitoring the peritoneum health of a patient based on changes to one or more fluid parameters.

FIG. 2 shows a flow chart of a method of monitoring the peritoneum health of a patient based on multiple fluid parameters received at different times.

FIG. 3 shows a flow chart of a method of monitoring the peritoneum health of a patient based on fluid parameters received from multiple dialysis sessions.

FIG. 4 is a system for monitoring the peritoneum health of a patient.

FIG. 5 shows a flow chart of a method of monitoring the peritoneum health of a patient based on combinations of fluid parameters.

### DETAILED DESCRIPTION OF THE INVENTION

Unless defined otherwise, all technical and scientific terms used generally have the same meaning as commonly understood by one of ordinary skill in the art.

The articles “a” and “an” are used to refer to one or to over one (i.e., to at least one) of the grammatical object of the article. For example, “an element” means one element or over one element.

The terms “alert,” “providing an alert,” or to “provide an alert” refer to any audio, visual, or tactile indication of a particular state of a system or patient.

The term “at least” refers to no less than or at the minimum. For instance, “at least one” could be one or any numbers more than one.

The term “bacteria concentration test” can refer to a determination of bacterial concentration in a sample. The bacterial concentration can be measured by turbidity, viable cell count, wet mass, and other methods known to a person of skill.

The term “bacteria testing” refers to a determination as to a type and amount of bacteria in a fluid or patient.

A “catheter” is a fluid connector or tube inserted into the body of a patient for introduction and removal of fluids to and from the patient.

The term “catheter kinking” refers to a line in a catheter bending in such a way as to impede fluid flow.

The term “catheter occlusion” or “catheter obstruction” refers to a blockage of a line in a catheter.

The term “catheter patency” refers to the ability of fluids to pass into and out of a catheter.

The term “change in peritoneum health” refers to any acute or chronic changes to the peritoneum of the patient, including the onset of disease, worsening of disease, or any other changes.

The terms “combination” or “combined” refer to using each of two or more fluid parameters in making a determination of a particular state or variable.

A “combined effluent and infusion line” is a single fluid line through which peritoneal dialysate can be both infused into and removed from a peritoneal cavity of a patient.

The term “communication” refers to an electronic or wireless link between two components.

The term “sensor suite” refers to a group of one or more sensors monitoring different fluid parameters.

The terms “comparing,” to “compare,” or “comparison” refer to determining the differences, if any, between two values or parameters.

The term “comprising” includes, but is not limited to, whatever follows the word “comprising.” Use of the term indicates the listed elements are required or mandatory but that other elements are optional and may be present.

The term “computer implemented” refers to a process or set of steps carried out by a processor, computer, or any other electronic system.

The terms “concentration” and “solute concentration” refers to an amount of a solute dissolved in a given amount of a solvent.

The term “conductivity sensor” refers to any component capable of measuring the electrical conductance or the electrical resistance of a fluid.

The term “consisting of includes and is limited to whatever follows the phrase “consisting of” The phrase indicates the limited elements are required or mandatory and that no other elements may be present.

The term “consisting essentially of includes whatever follows the term “consisting essentially of and additional elements, structures, acts or features that do not affect the basic operation of the apparatus, structure or method.

The term “correlation between fluid flow rate and pressure” refers to the pressure exerted by a fluid at particular flow rate in a conduit or system. As the flow rate increases, the pressure also increases. The increase in pressure as a function of the increase in flow rate is the correlation between flow rate and pressure.

The term “cycle” or “peritoneal dialysis cycle” refers to the infusion of peritoneal dialysate into a patient, a dwell of the peritoneal dialysate within the peritoneal cavity of the patient, and the removal of the peritoneal dialysate from the peritoneal cavity of the patient. The process of filling and then draining your abdomen can also be seen as an “exchange” used and clean fluids. However, the number, length, and timing of “cycles” or “exchanges” are non-limiting. For example, Continuous Ambulatory Peritoneal Dialysis (CAPD) and Continuous Cycling Peritoneal Dialysis (CCPD) may occur on different schedules, but the process of filling and then draining the peritoneal cavity can be referred to as “cycles” for both CAPD and CCPD. As such, the term is “cycle” or exchange refers to any particular dialysis schedule or type of dialysis.

The term “cycle number” refers to an order of a particular cycle in a peritoneal dialysis session. For example, cycle number one is the first cycle of a session.

The term “detachable” relates to any component of that can be separated from a system, module, cartridge or any component of the invention. “Detachable” can also refer to a component that can be taken out of a larger system with minimal time or effort. In certain instances, the components can be detached with minimal time or effort, but in other instances can require additional effort. The detached component can be optionally reattached to the system, module, cartridge or other component.

The terms “determining” and “determine” refer to ascertaining a particular state of a system or variable(s).

The term “dialysate effluent sensor” refers to a sensor used to measure one or more fluid parameters in used peritoneal dialysate removed from the peritoneal cavity of a patient.

The term “dwell time” refers to the amount of time elapsed between infusion of peritoneal dialysate into a patient and drainage of the peritoneal dialysate out of the patient.

The term “effluent boiling point elevation” refers to the increase in the boiling point of fluid removed from the peritoneal cavity of a patient as compared to the boiling point of pure water.

The term “effluent clarity” refers to the percentage of light shined on a fluid removed from the peritoneal cavity of a patient that passes through the fluid.

The term “effluent color” refers to the wavelength(s) of light absorbed or transmitted by a fluid removed from the peritoneal cavity of a patient.

The term “effluent dextrose concentration” refers to the amount of dextrose dissolved in a given amount of fluid removed from the peritoneal cavity of a patient.

The term “effluent freezing point depression” refers to the decrease in the freezing point of fluid removed from the peritoneal cavity of a patient as compared to the freezing point of pure water.

The term “effluent glucose concentration” refers to the amount of glucose dissolved in a given amount of fluid removed from the peritoneal cavity of a patient.

The term “effluent line” refers to a fluid connector for removing fluid from a peritoneal cavity of a patient. The term effluent line can also refer to a combined infusion and effluent line.

The term “effluent pH” refers to the negative log of the concentration of hydrogen ions in fluid removed from the peritoneal cavity of a patient.

The term “effluent refractive index” refers to a degree to which light is bent while travelling through fluid removed from the peritoneal cavity of a patient.

The term “effluent solute concentration” refers to a concentration of a solute within fluid removed from a peritoneal cavity of a patient.

The term “effluent temperature” refers to the temperature of fluid removed from the peritoneal cavity of a patient.

The term “effluent urea concentration” refers to the amount of urea dissolved in a given amount of fluid removed from the peritoneal cavity of a patient.

The term “execute” means to carry out a process or series of steps.

The term “fibrosis” refers to a thickening or scarring of tissue in a patient.

A “fluid” is a liquid substance optionally having a combination of gas and liquid phases in the fluid. Notably, a liquid can therefore also have a mixture of gas and liquid phases of matter.

The terms “fluidly connectable,” “fluidly connected,” “fluid connection” “fluidly connectable,” or “fluidly connected” refer to the ability to pass fluid, gas, or mixtures thereof from one point to another point. The two points can be within or between any one or more of compartments, modules, systems, and components, all of any type.

The term “flow fluctuations” refers to changes in fluid flow rate while infusing a fluid into a patient.

The term “flow rate” refers to the volume of fluid moving through a conduit or system per unit time.

A “fluid flow sensor” is a sensor that measures the fluid flow rate or volume of fluid infused to a patient.

A “fluid parameter” is any sensed characteristic of a fluid, including temperature, pressure, concentration, color, or any other characteristic.

The term “fluid pressure” refers to the force exerted by a fluid within a fluid line.

The term “fluid volume removed” or “fluid removal volume” refers to the net volume of fluid removed from a patient during a peritoneal dialysis cycle. The fluid removal volume is equal to the difference between the amount of peritoneal dialysate infused into the patient and the amount of effluent removed from the patient with full draining.

The term “infection” refers to any virus or bacteria in a patient's tissue that is not normally in the patient's tissue.

The term “infection sensor” can refer to a sensor that monitors infection status by measuring one or more characteristics indicating changes of the infection status, such as pH, color, clarity, temperature, bacterial concentration, and white cell concentration of a sample.

“Inflammation” is a protective biological response of body tissues to harmful stimuli, such as pathogens, damaged cells, or irritants, involving immune cells, blood vessels, and molecular mediators.

The term “infusing” or to “infuse” a fluid refers to the movement of peritoneal dialysate into the peritoneal cavity of a patient.

An “infusion line” is a fluid line for carrying peritoneal dialysate into a body cavity or part of a patient such as a peritoneal cavity. The term infusion line can also refer to a combined infusion and effluent line.

The term “instructions” refers to digital information that, when read or executed by a computer, processor, or system, cause the computer, processor, or system to carry out a series of steps.

An “integrated cycler” is a component for movement of fluid into and out of the peritoneal cavity of a patient, wherein the integrated cycler forms a part of an overall system. For example, the integrated cycler can be contained in a housing with other components used for peritoneal dialysis and be in fluid and electrical connection with desired components.

The term “intraperitoneal pressure” refers to the fluid pressure within the peritoneal cavity of a patient.

“Kt/V” refers to a number used to quantify dialysis treatment adequacy. K stands for dialyzer clearance of urea, t stands for dialysis time. Kt is clearance multiplied by time, representing the volume of fluid completely cleared of urea during a single treatment. V stands for the volume of water a patient's body contains.

The term “machine-readable storage medium” refers to any electronic device capable of storing information in a digital format for reading by a computer, processor, or other electronic device.

The term “membrane transfer efficiency” refers to the ability of water or one or more solutes to travel through a semi-permeable membrane, such as the peritoneal membrane of a patient.

The term “membrane wearout” refers to a condition of a patient wherein the peritoneal membrane is no longer capable of effectively transferring fluid and solutes.

The term “monitoring” or to “monitor” refers to determining a status of a system or patient over time.

The term “osmolality” can refer to a count of the number of particles in a solution. “Total osmolality” is the sum of the osmolalities of all the solutes present in the solution.

The term “osmolality sensor” can refer to a sensor that monitors osmolality of a sample by measuring one or more characteristics indicating changes of the osmolality, such as a refractive index, boiling point elevation, and freezing point depression.

An “osmotic agent” is a substance dissolved in water capable of driving a net movement of water by osmosis across a semi-permeable membrane due to concentration differences of the osmotic agent on each side of the semi-permeable membrane.

The term “osmotic agent concentration” refers to the amount of an osmotic agent dissolved in a fluid per unit of volume.

A “patient” or “subject” can refer to a member of any animal species, preferably a mammalian species, optionally a human. The subject can be an apparently healthy individual, an individual suffering from a disease, or an individual being treated for a disease.

The term “perform” means to carry out a set of steps or operations.

“Peritoneal dialysis” is a therapy wherein a dialysate is infused into the peritoneal cavity, which serves as a natural dialyzer. In general, waste components diffuse from a patient's bloodstream across a peritoneal membrane into the dialysis solution via a concentration gradient. In general, excess fluid in the form of plasma water flows from a patient's bloodstream across a peritoneal membrane into the dialysis solution via an osmotic gradient. Once the infused peritoneal dialysis solution has captured sufficient amounts of the waste components the fluid is removed. The cycle can be repeated for several cycles each day or as needed.

The term “peritoneal dialysis cycler” or “cycler” can refer to components for movement of fluid into and out of the peritoneal cavity of a patient, with or without additional components for generating peritoneal dialysate or performing additional functions.

The term “peritoneal dialysis filtrate” or “filtrate” can refer to fluid removed from the peritoneal cavity of a patient during peritoneal dialysis therapy.

A “peritoneal dialysis session” is a set of peritoneal dialysis cycles performed over a time period as part of ongoing therapy. The peritoneal dialysis session can last a day or more, and can include any number of cycles.

“Peritonitis” is an inflammation of the peritoneal membrane of a patient. Peritonitis is considered to be a major complication of peritoneal dialysis. The term “night enteric peritonitis” refers to a peritonitis relating to or affecting intestines.

“Peritonitis sensor” can be a sensor that monitors peritoneum health based on one or more characteristics, such as pH, clarity, temperature, color, bacterial concentration, and white cell concentration of a sample.

The term “peritoneum” refers to the lining of the abdominal cavity in a patient.

The term “peritoneum health” refers to any physiological factors relating to the peritoneum affecting the overall health of a patient. Peritoneum health can refer, without limitation, to infection, peritonitis, or a failing peritoneum.

The term “peritoneum membrane health” can refer to the transport status of the peritoneum membrane. It is known that long-term peritoneal dialysis may affect the peritoneum membrane health, leading to peritoneal membrane failure.

The term “pH of a peritoneal dialysate input” refers to the hydrogen ion concentration in fluid infused into the peritoneal cavity of a patient.

The term “pH of a peritoneal dialysis filtrate” refers to the hydrogen ion concentration in a fluid removed from the peritoneal cavity of a patient.

The term “pH sensor” refers to any component capable of measuring the hydrogen ion concentration in a fluid.

The term “positioned” refers to the location of a component.

The term “predetermined threshold” refers to a value for a parameter, set before analysis to which the analyzed parameter can be compared. Whether the analyzed parameter exceeds or does not exceed the predetermined threshold can direct or cause some action to be taken.

The term “pressure fluctuations” refer to changes in fluid pressure while infusing a fluid into a patient.

The term “pressure sensor” refers to any component capable of determining the force exerted by a fluid.

The term “pressure to infuse peritoneal dialysate” refers to the pressure exerted by a fluid while infusing the fluid into a patient at a specified flow rate.

The term “prior history of a patient” refers to the dialysis parameters used and the resulting patient parameters or dialysis results from one or more previous dialysis sessions.

The term “processor” is a broad term and is to be given its ordinary and customary meaning to a person of ordinary skill in the art. The term refers without limitation to a computer system, state machine, processor, and the like designed to perform arithmetic or logic operations using logic circuitry that responds to and processes the basic instructions that drive a computer. In any embodiment of the first, second, third, and fourth invention, the terms can include ROM (“read-only memory”) and/or RAM (“random-access memory”) associated therewith.

The term “protein composition” refers to the type and amount of proteins in a fluid.

The term “protein leakage” refers to the degree to which proteins are transferred through the peritoneal membrane of a patient during peritoneal dialysis.

The term “providing a fluid bolus” or to “provide a fluid bolus” refers to transferring a volume of fluid to a patient through a catheter.

The term “receiving” or to “receive” means to obtain information from any source.

The term “refractive index” of a substance can refer to the ratio of the velocity of light in a vacuum to its velocity in the substance. Refractive index depends on composition, concentration (e.g. dry substance) and temperature of the substance. “Refractive index changes of dextrose” can refer to changes of the refractive index of dextrose.

The term “refractive index sensor” refers to a device that detects or measures a refractive index, and records, indicates or otherwise responds to it.

The term “sampling reservoir” refers to a container for collecting a portion of a fluid for analysis of the fluid separate from the rest of a system.

A “sensor” is a component capable of determining one or more states of one or more variables in a system. The term “sensor suite” can refer to one or more sensors for measuring one or more characteristics of a sample.

A “solute” is a substance dissolved in, or intended to be dissolved in, a solvent.

The term “storing” or to “store” refers to saving electronic data or information in a machine readable medium.

The term “toxin transfer” refers to the ability of a patient to pass toxins through the peritoneal membrane during peritoneal dialysis.

The terms “trend” or “trending” refer to determining changes in values for one or more parameters over time.

The term “ultrafiltrate sensor” refers to a sensor used to measure one or more fluid parameters in an ultrafiltrate removed from a patient after a peritoneal dialysis cycle.

The term “ultrafiltrate transfer efficiency” refers to the volume and rate that fluid is removed from a patient during a cycle, taking into account all dialysis parameters.

The term “ultrafiltration volume” refers to the net amount of fluid removed from a patient during a dialysis session.

The term “URR” can refer to urea reduction ratio wherein the reduction in urea is a result of dialysis. The URR is one measure of how effectively a dialysis treatment removed waste products from the body.

The term “white cell count” refers to the number of white blood cells in a given volume of fluid. The term “white cell concentration” equals to the white cell count divided by the given volume.

### Catheter and Peritoneum Health Monitoring

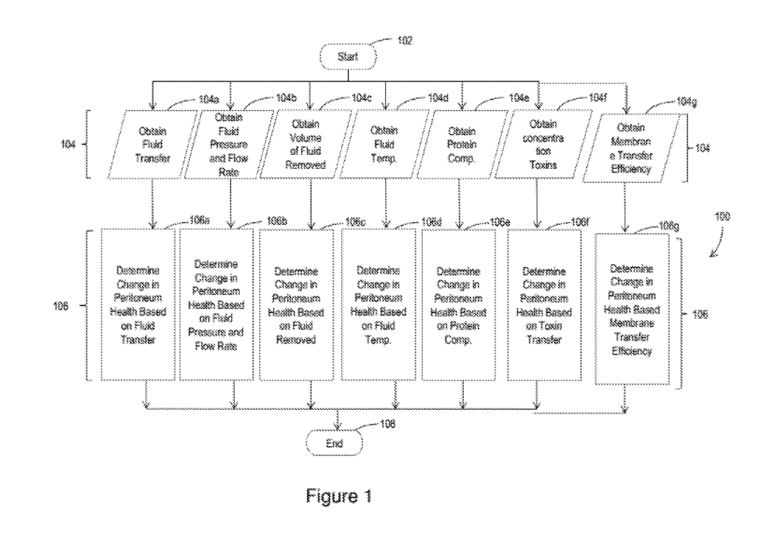

FIG. 1 is a flowchart of a computer implemented method 100 for receiving one or more fluid parameters during a peritoneal dialysis session to determine a change in peritoneum health of a patient based on the obtained one or more fluid parameters. The computer implemented method 100 can be performed using a system configured to monitor fluid parameters during a peritoneal dialysis session, such as the system 400 of FIG. 4. Instructions for carrying out the method 100 illustrated in FIG. 1 can be stored in a machine-readable storage medium. A processor in a dialysis machine can execute the instructions to perform the computer implemented method 100.

The method 100 can begin in operation 102. A peritoneal dialysis session can already be underway. In operation 104, one or more fluid parameters can be received into the system during a peritoneal dialysis session. The fluid parameters can be stored in a database or any other machine-readable storage medium. The fluid parameters can be received into the system as input into an input/output interface of the system, or automatically received by the system from one or more sensors in communication with a processor of the system.

Multiple instances of operation 104 are depicted in FIG. 1. For example, in operation 104a, a net or given fluid transfer volume can be received into the system. The net fluid transfer volume can be calculated by measuring the difference between the volume of peritoneal dialysate infused into the patient and the volume of filtrate removed from the patient to obtain an ultrafiltration transfer efficiency. A given fluid transfer volume is any volume of fluid infused into or drained out of the patient. Fluid flow sensors in the infusion and effluent line of a cycler can measure the volume infused into and removed from the patient. In operation 104b, a fluid pressure measurement can be received into the system from a pressure sensor located in an infusion line. The pressure sensor can measure the pressure required to infuse fluid into the peritoneal cavity of the patient. The fluid flow rate of the fluid into the peritoneal cavity is also obtained from a fluid flow sensor in the infusion line. In operation 104c, a given or net volume of effluent removed from a peritoneal cavity of a patient during one or more cycles of the current session can be measured using a fluid flow sensor of the system and received into the computing device. As another example, in operation 104d, a temperature of effluent removed during the current PD session can be measured using a temperature sensor along the effluent line, and/or a temperature of peritoneal dialysate input temperature can be measured using a temperature sensor along the infusion line. In operation 104e, a protein composition in the peritoneal dialysis filtrate can be obtained using an ultrafiltrate sensor using the Coomassie, Bicinchoninic acid, Pierce or similar chromogenic protein assay technology. All of these assays require combining the protein solution with a dye or other reagent to produce a colored solution that can be measured using UV/Vis spectrophotometry. Alternatively, the total protein composition can be measured directly by UV/Vis spectrophotometry at 280 nm without need of additional reagents. In operation 104f, a concentration of one or more toxins, such as creatinine, uric acid, or urea in the peritoneal dialysis filtrate can be measured using an ultrafiltrate sensor including UV/Vis spectrophotometry, or other suitable assay to determine the toxin transfer efficiency. In operation 104g, a membrane transfer efficiency is calculated. The membrane transfer efficiency can be calculated from a given or net ultrafiltration volume, taking into account the cycle number, dwell time, and dialysate composition. The membrane transfer efficiency can also be determined by measuring the effluent conductivity or effluent solute concentration in the dialysate at multiple points in time during a dwell period.

The method 100 can proceed to operation 106. In operation 106, a change in peritoneum health can be determined based on the one or more fluid parameters. Multiple instances of operation 106 are depicted in FIG. 1. For example, in operation 106a, a change in peritoneum health is determined using the fluid transfer obtained in operation 104a. A low given or net fluid transfer volume could indicate failing peritoneum health. In operation 106b, a change in the fluid pressure at a particular fluid flow rate can be used to determine changes in the peritoneum health. The system can correlate the pressure and fluid flow rate across multiple sessions to determine the pressure of infusing fluid into the patient at a particular fluid flow rate. An increase in the pressure necessary to infuse fluid into the patient at a particular fluid flow rate may indicate peritonitis, which is an inflammation of the peritoneum and a major complication of peritoneal dialysis. Similarly, pressure fluctuations or flow fluctuations may indicate a partial or complete flow obstruction in the catheter. Additionally, an increase in the pressure necessary to infuse fluid into the patient at a particular fluid flow rate may indicate fibrosis formation in the catheter. Other catheter issues, such as catheter kinking, catheter wear-out, catheter occlusion, change in catheter position that impedes flow, or other defects can also be determined by an increase in pressure at a particular fluid flow rate. Although, some variation in the pressure/flow rate relationship may be expected, a difference of more than a set value could indicate a catheter problem. For example, an increase in pressure at a flow rate of more than 25% could indicate a problem with the catheter. Normal variation of 10%, can however, be expected. Increased intraperitoneal pressure can also indicate peritonitis, as higher pressure is correlated with night enteric peritonitis and higher patient mortality. Increases in intra-abdominal pressure can lead to abdominal hypertension, abdominal hernia, impaired dialysis effectiveness, strongly reduced ultrafiltration volume, and abdominal compartment syndrome. Studies have shown that there is no significant difference between intraperitoneal pressure and intra-abdominal pressure (Al-Hwiesh, et al., Peritoneal Dialysis International, Vol. 31, pp. 315-319). In one study of intraperitoneal pressures in patients with kidney disease, the mean intraperitoneal pressure 9.0±5.0 mmHg while supine, and 16.0±7.0 mmHg while erect in the dry state. In the filled state, the mean intraperitoneal pressure was found to be 12±2.0 mmHg while supine and 13±3.0 mmHg while erect. The pressure was found to rise 2 cm of H₂O for each liter of fluid infused into the patient. The system can measure intraperitoneal or intra-abdominal pressure and trend the pressure changes over e in either the dry or filled state. Using the trend of intraperitoneal or intra-abdominal pressure, the system can determine changes in peritoneum health of the patient, and issue an alert of the trend shows an increase in pressure over time. The system can also issue an alert if the patient's intra-abdominal or intraperitoneal pressure increases over the mean pressure by a predetermined threshold.

In operation 106c, a change in the peritoneum health can be determined by the change in the net or given fluid removal volume. The net or given fluid removal volume is a function of the dwell time, cycle number, osmotic agent concentration and membrane transfer efficiency. A drop in the net or given fluid removal volume could indicate a decrease in membrane transfer efficiency, which may indicate a failing peritoneum in the patient. In operation 106d, the effluent temperature is used to determine a change in peritoneum health. Increasing temperature in the effluent removed from the patient could indicate an infection in the peritoneum. In operation 106e, the protein composition of the filtrate or effluent is used to determine a change in peritoneum health. The protein composition and concentrations in the effluent can be trended over multiple sessions. An increase in the protein concentration in the effluent may indicate protein leakage, which is correlated with a failing peritoneum. In operation 106f, the toxin concentration, such as urea, creatinine, uric acid or other known uremic toxins, in the filtrate or effluent is used to determine a change in peritoneum health by determining the toxin transfer efficiency. An unexpected increase in the amount of proteins or other uremic toxin such as urea, creatinine, uric acid or other uremic toxin could indicate peritonitis. An unexpected decrease in protein, or other uremic toxin such as urea, creatinine, uric acid or other uremic toxin could indicate a failing peritoneum. In operation 106g, a change in the membrane transfer efficiency is used to determine a change in peritoneum health. A decrease in membrane transfer efficiency over multiple sessions could indicate membrane wear-out or a failing peritoneum. One of skill in the art will understand that additional fluid parameters not illustrated in FIG. 1 can also be determined. For example, the peritoneal dialysate effluent can undergo bacteria testing to determine if an infection or peritonitis is present. A white cell count can also be determined, which can indicate infection or peritonitis. Additional optical changes in the dialysate can also be detected to determine whether a catheter obstruction or occlusion is present, causing blockage of the catheter. A coulter counter, which determines the size and number of particles in a fluid can be used to measure the optical changes that may indicate a catheter occlusion. Alternatively, a refractive index meter or conductivity meter can be used to determine the makeup of particles in the peritoneal dialysate effluent. The osmotic agent concentration in the peritoneal dialysate effluent can be determined by a glucose sensor or a mini-spectrophotometer. One non-limiting example of a glucose sensor is the Enlite® glucose sensor from Medtronic, Inc. However, alternative glucose sensors can also be used. Glucose and/or dextrose concentration in the fluid can be monitored with a refractive index sensor, and used to determine the total osmolality of the peritoneal dialysate effluent. Total Osmolality is the sum of the osmolalities of all the solutes present in the peritoneal dialysate effluent. The boiling point elevation and/or freezing point depression of the peritoneal dialysate effluent can be used to determine the total osmolality of the peritoneal dialysate effluent. The effluent boiling point elevation refers to the increase in the boiling point of fluid removed from the peritoneal cavity of a patient as compared to the boiling point of pure water. “The effluent freezing point depression” refers to the decrease in the freezing point of fluid removed from the peritoneal cavity of a patient as compared to the freezing point of pure water. A higher than expected total osmolality of the peritoneal dialysis effluent, or a total osmolality of the peritoneal dialysis effluent trending higher can indicate a loss in membrane transfer efficiency. A urea sensor, can be used to determine the Kt/V for the treatment. Kt/V is a number used to quantify dialysis treatment adequacy, where K stands for dialyzer clearance of urea, t stands for dialysis time. Kt is clearance multiplied by time, representing the volume of fluid completely cleared of urea during a single treatment, and V is the volume of water a patient's body contains. In patients that combine peritoneal dialysis and hemodialysis, the Kt/V and residual kidney function can be combined to determine the combined Kt/V. Any urea sensor can be used to determine the urea concentration. A lower Kt/V could indicate a decrease in membrane transfer efficiency and a failing peritoneum. In a non-limiting embodiment, a urea sensor can use urease to convert urea to ammonium ions, which are then measured.

One of ordinary skill in the art will recognize that multiple fluid parameters can be monitored and/or obtained and analyzed to determine changes in peritoneum health. The method 100 can comprise any of the parameters, either alone or in combination. As a non-limiting example, a combination of one or more of membrane transfer efficiency from operation 104g, protein composition from operation 104e, and net fluid removal from operation 104c can more accurately determine peritoneal membrane wear out or a failing peritoneum than any parameter alone. A combination of one or more of the pressure to infuse fluid at a particular flow rate from operation 104b and the toxin concentration from operation 104f can be used to more accurately determine peritonitis. Alternatively, a combination of pressure and flow fluctuations can more accurately determine obstructions in the catheter. A combination of a net fluid removal from operation 104c and forward or reverse fluid transport measurements from operation 104a can more accurately measure changes in peritoneal membrane health, as clearance decreases for a given URR or urea concentration. URR stands for urea reduction ratio, meaning the reduction in urea as a result of dialysis. Improve the peritoneal membrane health is important in peritoneal dialysis, because the peritoneal membrane in decreased health can negatively affects the transport across the membrane. A combination of one or more of refractive index, boiling point elevation, and/or freezing point depression can more accurately determine the total osmolality of the fluid as compared to a single parameter alone. One of skill in the art will recognize that various combinations of the fluid parameters can be used to more accurately assess changes in peritoneum health. In operation 108, the method 100 can end. If a worsening of peritoneum health or catheter status is determined in operation 106, an alert can be issued to the patient or health care professional indicating infection, catheter problems, or a failing peritoneum. The alert can indicate the need to intervene by altering the dialysate composition, changing the catheter, or transferring the patient to hemodialysis.

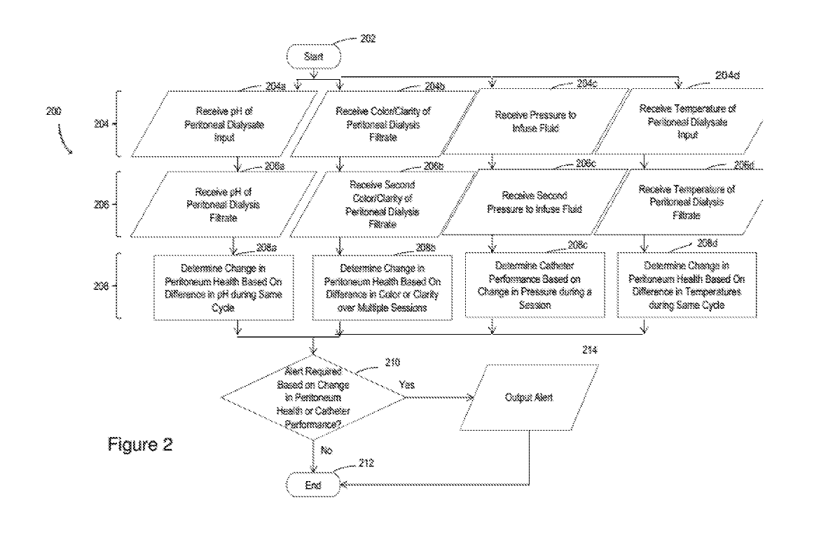

FIG. 2 is a flowchart of a computer implemented method 200 for receiving one or more fluid parameters during a peritoneal dialysis session to determine a change in peritoneum health or catheter performance based on the obtained one or more fluid parameters. The method 200 can be performed using a system configured to monitor fluid parameters during a peritoneal dialysis session, such as the system 400 of FIG. 4. Instructions for carrying out the method 200 illustrated in FIG. 2 can be stored in a machine-readable storage medium. A processor in a dialysis machine can execute the instructions to perform the method 200. The method 200 can begin in operation 202. A peritoneal dialysis session can already be underway.

In operations 204 and 206, fluid parameters can be obtained from the one or more sensors of the system. Multiple instances of operations 204 and 206 are depicted in FIG. 2. For example, in operation 204a, a pH of peritoneal dialysate input can be received during a fill portion of a cycle using a pH sensor along an infusion line. In operation 206a, a pH of peritoneal dialysis filtrate can be received during a drain portion of a cycle using a pH sensor along an effluent line. Alternatively, a small portion of filtrate can be removed from the patient during a dwell period to determine the pH of the peritoneal dialysate at any time during therapy. As another example, in operation 204b, a color and/or clarity of a peritoneal dialysis filtrate from a first session can be received into the system and can be measured by a dialysate effluent sensor. In operation 206b, a color and/or clarity of a peritoneal dialysis filtrate from a second session can be received into the system. The color spectrum of the peritoneal dialysate filtrate can be determined using a spectroscope. The spectroscope can be attached to the effluent line of a cycler to determine the color and clarity spectrophotometrically. Alternatively, a sample of filtrate from the effluent line can be removed and analyzed using an off-line or integrated spectroscope to determine the color and clarity of the filtrate. In operation 204c, the pressure to infuse fluid into a patient can be determined during a first cycle, or from prior sessions. In operation 206c, a pressure to infuse fluid into a patient can be determined from pressure sensors in the infusion line. In operation 204c, a temperature of the peritoneal dialysis input can be measured with a dialysate effluent sensor, such as a temperature sensor in the infusion line. In operation 206d, a temperature of the peritoneal dialysis filtrate can be determined from temperature sensors in the effluent line. FIG. 2 shows both receiving two different fluid parameters (operations 204a, 206a, and 204d, 206d) and receiving two instances of the same fluid parameter (operations 204b, 206b, and 204c, 206c).

The method 200 can proceed to operation 208. In operation 208, a change in peritoneum health can be determined based in the received fluid parameters. Multiple instances of operation 208 are depicted in FIG. 2. For example, in operation 208a, the pH of the peritoneal dialysis filtrate can be compared with the pH of the peritoneal dialysate input and the change in peritoneum health can be determined based on a difference in pH of the peritoneal dialysis filtrate and the pH of the peritoneal dialysate input. A drop in pH of the filtrate during the dwell time could indicate infection of the peritoneum. As another example, in operation 208b the effluent color and/or clarity of the peritoneal dialysis filtrate of the first session can be compared with the effluent color and/or clarity of the peritoneal dialysis filtrate of the second session. The color and clarity of the effluent can change due to fibrin in the peritoneum or increased triglycerides in the filtrate. The color and clarity of the fluid could indicate infection in the peritoneum of the patient. In response to a change in the color spectrum of the fluid or clarity of the fluid, an alert can be issued to a health care professional indicating a possible infection.

Additionally, changes to the color and clarity of the fluid can be an early sign of peritoneum membrane wear out. In operation 208c catheter patency can be determined based on a change in the pressure necessary to infuse fluid into a patient. A sudden increase in pressure could indicate an occlusion of a fluid line, such as an object or person sitting on the line. An increase in pressure could also indicate that the catheter inlet is lying against the peritoneal tissues and has become occluded. In operation 208d, the difference in the temperature of the peritoneal dialysate input and the peritoneal dialysate filtrate can be determined. An increase in temperature during a cycle could indicate an infection. One of skill in the art will recognize that the method can comprise multiple combinations of the fluid parameters to determine changes in peritoneum health. As a non-limiting example, a combination one or more of the color or clarity measured in operations 204b and 206b, the change in pH measured in operations 204a and 206a, a change temperature measured in operations 204d and 206d, as well as bacteria testing and white cell count can be used to more accurately determine the presence of infection and/or peritonitis as compared to any single parameter alone. A combination of one or more of the flow rate and the pressure necessary to infuse fluid into a patient from operations 204c and 206c as well as optical changes from operations 204b and 206b can more accurately determine catheter obstructions than any single parameter alone. A combination of optical changes to the peritoneal dialysate effluent, pH, and color changes can more accurately determine changes to the peritoneum health than any single parameter alone. One of skill in the art will recognize that various other combinations of the disclosed fluid parameters can be used to more accurately assess changes in peritoneum health.

The method 200 can proceed to operation 210. In operation 210, a determination is made whether an alert is desired based on the change in peritoneum health determined in operation 208. For example, in the pH comparison example, if the pH of the peritoneal dialysis filtrate is lower than the pH of the peritoneal dialysate input, and if the difference in pH of the peritoneal dialysis filtrate and the pH of the peritoneal dialysate input is greater than a predetermined value, an alert may be desirable and the method 200 can proceed to operation 214. As another example, in the effluent color/clarity example, if the color and/or clarity of the peritoneal dialysis effluent changes between the first session and the second session by more than a predetermined threshold, then a determination is made that an alert is desirable, and the method 200 can proceed to operation 214. Alternatively, if the pressure to infuse fluid into the patient increases beyond a predetermined threshold, a determination is made that an alert is desirable, and the method 200 can proceed to operation 214. In addition to an alert, if the system determines that the catheter may be occluded by tissue in the peritoneal cavity, the system can provide a fluid bolus through the catheter to pulse the catheter and move the catheter within the peritoneal cavity or dislodge an obstruction at the catheter outlet. The system can begin testing the pressure again after providing the fluid bolus to determine if the catheter problem has been resolved.

In operation 214, an alert regarding peritoneum health can be output from the system. The alert can be output can be displayed to a user, can be printed, can be electronically communicated to the patient's doctor through wireless or wired communication, or otherwise communicated. The method 200 can proceed to operation 212 and the method 200 can end. If, in operation 210, a determination is made that an alert is not desired, the method 200 can proceed to operation 212 and the method 200 can end.

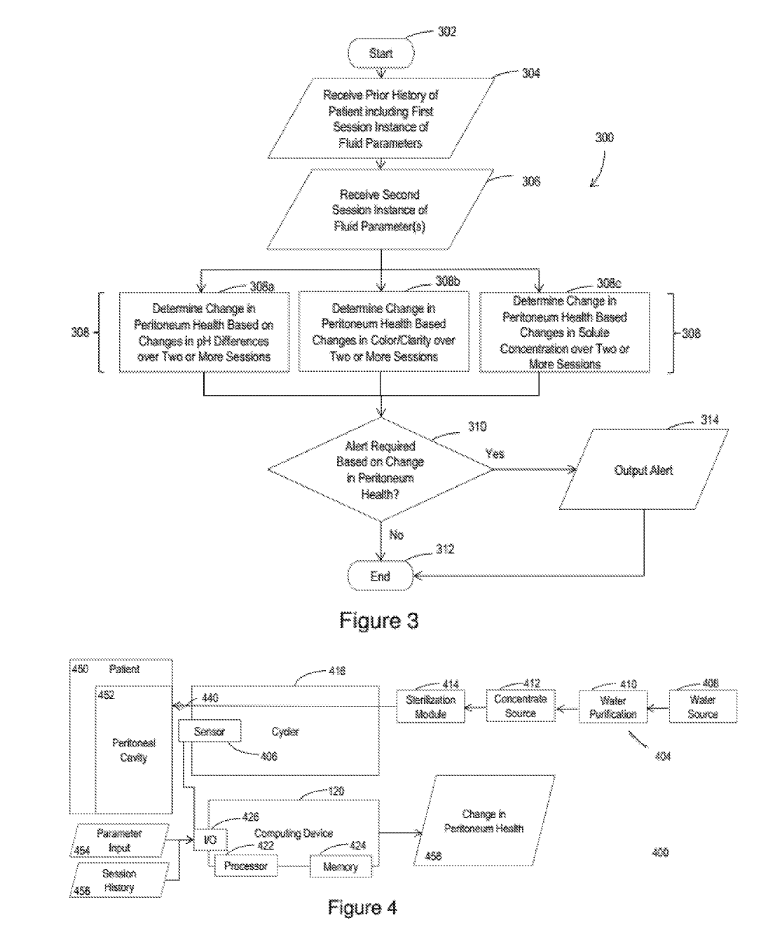

FIG. 3 is a flowchart of a computer implemented method 300 for obtaining one or more fluid parameters during a peritoneal dialysis session to determine a change in peritoneum health of a patient based on the obtained one or more fluid parameters. The method 300 can be performed using a system configured to monitor fluid parameters during a peritoneal dialysis session, such as the system 400 of FIG. 4. Instructions for carrying out the method 300 illustrated in FIG. 3 can be stored in a machine-readable storage medium. A processor in a dialysis machine can execute the instructions to perform the method 300.

The method 300 can begin in operation 302. A peritoneal dialysis session can be already underway, or can be in the process of initiating. In operation 304, prior history of a patient can be received into the system configured to monitor fluid parameters during a peritoneal dialysis session and stored in a machine-readable storage medium. For example, the prior history of the patient can be received as input into an input output interface of the system. As another example, the prior history of the patient can be received from memory of the system. Alternatively, the prior history of the patient can be received electronically from the patient's electronic medical records. The prior history of the patient can include fluid parameters monitored during a previous session using the one or more sensors, and/or parameters derived from the monitored parameters.

For example, the prior history of the patient can include a pH of a peritoneal dialysate input from a previous session and a pH of a peritoneal dialysis filtrate from the previous session. The prior history of the patient can include a difference between the pH of a peritoneal dialysis filtrate and the pH of a peritoneal dialysate input. As another example, the prior history of the patient can include an effluent color and/or an effluent clarity of the peritoneal dialysis effluent from the previous session. The method 300 can proceed to operation 306.

In operation 306, one or more fluid parameters from the current peritoneal dialysis session can be received from the sensors of the system. For example, a pH of a peritoneal dialysate input of the current session can be received from a pH sensor positioned in an infusion line of the system. A pH of a peritoneal dialysis filtrate of the current session can be received from a pH sensor positioned in an effluent line of the system. Fluid parameters can be derived from obtained fluid parameters. For example, a processor of the system can derive a difference in pH of the peritoneal dialysis filtrate and the pH of the peritoneal dialysate input of the current session. As another example of fluid parameters from the current peritoneal dialysis session that can be received from the sensors of the system, a color and/or clarity of the peritoneal dialysis filtrate from a cycle of the current session can be received from an integrated or external spectroscope. Effluent ionic solute concentrations can be sensed by conductivity sensors or ion selective electrodes positioned in the effluent line. The method 300 can proceed to operation 308.

In operation 308, a change in peritoneum health can be determined based on trends across two or more peritoneal dialysis sessions. That is, a change in peritoneum health can be determined based on changes in the one or more fluid parameters over the previous session (e.g., first session) and the current session (e.g., second session). One of skill in the art will understand that any number of sessions can be included in the prior history of the patient. The patient parameters can be trended over the multiple sessions to determine ongoing longer term changes in each parameter. The health of the peritoneum or catheter can be monitored by monitoring changes in the trends of parameters.

Multiple instances of operation 308 are depicted in FIG. 3. For example, in operation 308a a change in peritoneum health can be determined based on a change in the difference in pH of the peritoneal dialysis filtrate and the pH of the peritoneal dialysate input of the current session (e.g., second session) and a difference between the pH of a peritoneal dialysis filtrate and the pH of a peritoneal dialysate input of a previous session (e.g., first session), or changes to a longer term trend of pH. If the prior history of the patient does not contain the difference between the he pH of a peritoneal dialysis filtrate and the pH of a peritoneal dialysate input of a previous session, the processor of the system can derive the difference from the parameters of the prior history of the patient. The processor of the system can derive the difference between the he pH of a peritoneal dialysis filtrate and the pH of a peritoneal dialysate input of the previous session.

In operation 308b, a change in peritoneum health can be determined based on a change in the difference in color and/or clarity of the peritoneal dialysis effluent from one or more previous sessions to the current session, or a longer term trend in the color and clarity of the effluent. In operation 308c, a change in peritoneum health can be determined based on changes in solute concentrations in the peritoneal dialysate filtrate from one or more previous sessions to the current session. The changes in solute concentrations as measured at various times during a dialysis session can be used to calculate the membrane transfer efficiency of the peritoneum. A decrease in membrane transfer efficiency can indicate a failing peritoneum. One of skill in the art will recognize that changes in peritoneum health can be determined based on a combination of parameters. As a non-limiting example, a combination of changes in the difference in pH of the peritoneal dialysis filtrate and the pH of the peritoneal dialysate input over multiple sessions as determined in operation 308a, changes in color or clarity as determined in operation 308b, and changes in solute concentrations over one or more previous sessions as determined in operation 308c can more accurately determine changes in peritoneum health than single parameters alone. The method can comprise any combination of fluid parameters measured over two or more sessions to determine a change in peritoneum health. The method 300 can proceed to operation 310.

In operation 310, a determination is made whether an alert is desired based on the change in peritoneum health determined in operation 308. For example, in the pH comparison example, if the pH of the peritoneal dialysis filtrate is lower than the pH of the peritoneal dialysate input, and if the difference in pH of the peritoneal dialysis filtrate and the pH of the peritoneal dialysate input is greater than a predetermined value, an alert may be desirable and the method 300 can proceed to operation 314. The differences in the pH of the dialysis input and filtrate can be trended over multiple sessions. A trend showing an increase in the pH drop during the dwell period over multiple sessions could indicate an infection in the peritoneum. As another example, in the peritoneal effluent color/clarity example, if the color and/or clarity of the peritoneal dialysis effluent changes between the first session and the second session by more than a predetermined threshold, then a determination is made that an alert is desirable, and the method 300 can proceed to operation 314. The changes to the color and clarity of the effluent can be trended over multiple sessions. A trend showing an increase in cloudiness over time could indicate an infection in the peritoneum. As yet another example, in the solute concentration example, small amounts of filtrate can be removed from the patient at multiple times during a cycle to calculate the membrane transfer efficiency. A decrease in the membrane transfer efficiency trend over multiple sessions could indicate a failing peritoneum.

In operation 314, an alert regarding peritoneum health can be output from the system. The alert can be output can be displayed to a user, can be printed, can be electronically communicated to the patient's doctor, or otherwise communicated. The method 300 can proceed to operation 312 and the method 300 can end. If, in operation 310, a determination is made that an alert is not desired, the method 300 can proceed to operation 312 and the method 300 can end.

FIG. 4 shows a system 400 for receiving one or more fluid parameters during a peritoneal dialysis session determine a change in peritoneum health of a patient 450 based on the obtained one or more fluid parameters. One or more fluid parameters can be obtained by the system 400 during the peritoneal dialysis session such as before, during, or after a cycle. The one or more fluid parameters can be analyzed. The health of a patient's peritoneum can be determined based on the analysis.

The system 400 can include a combined peritoneal dialysate infusion and effluent line 440, referred to herein as a peritoneal dialysate effluent line, a peritoneal dialysate generation flow path 404, at least one sensor 406 positioned in one or both of the peritoneal dialysate effluent line 440 and the peritoneal dialysate generation flow path 404, and a computing device 420. Alternatively, separate peritoneal dialysate infusion and effluent lines can be used. One of skill in the art will understand that any number of sensors 406 can be included at multiple positions in the system 400. The sensor 406 can include a sensor suite for monitoring multiple fluid parameters. For example, a sensor suite can include one or more pressure sensors, one or more fluid flow sensors, one or more ultrafiltrate sensors, which measure fluid parameters in the ultrafiltrate removed from a patient 450; and/or one or more dialysate effluent sensors that measure fluid parameters in the effluent removed from a patient 450. Additional sensors, including pH sensors, conductivity sensors, or other sensors can be included in the sensor suite. Any one or more of the sensors in the sensor suite can be included as part of the integrated peritoneal dialysis cycler 416, or as external components. The peritoneal dialysate effluent line 440 can be fluidly connected to a waste reservoir to collect effluent. Alternatively, the peritoneal dialysate effluent line 440 can be fluidly connected to a sampling reservoir to remove small samples of the filtrate for off-line analysis. The sampling reservoir can be detachable from the cycler 416 to allow the removed filtrate to be tested by any sensors or components not included in the effluent line 440.

Alternatively, the sample can be diverted directly to standalone system (not shown), such as a blood analyzer for analysis. Blood analyzers can determine several fluid characteristics, which can be included in the system. One non-limiting example of a standalone analyzer is the Stat Profile® Critical Care Xpress analyzer by Nova Biomedical, however any analyzer can be used. The standalone analyzer can be in communication with the processor 422 or computing unit of the system 400 to provide the system 400 with the results of the analysis. Specialized tubing with a T-junction or a valve can be used to divert a volume of fluid to a standalone analyzer.

The peritoneal dialysate generation flow path 404 can include a separate infusion line (not shown) or the effluent line 440 can be used for infusion of fluid. The peritoneal dialysate generation flow path 404 can include a water source 408, one or more water purification modules 410, a concentrate source 412, a sterilization module 414, and an integrated cycler 416. The concentrate source 412 can contain one or more solutes. The water source 408, water purification module 410, concentrate source 412, sterilization module 414, and integrated cycler 416 can be fluidly connectable to the peritoneal dialysate generation flow path 404.

The water source 408 can be a non-purified water source, such as tap water, wherein the water from the water source 408 can be purified by the system 400. A non-purified water source can provide water without additional purification, such as tap water from a municipal water source, water that has undergone level of purification, but does not meet the definition of “purified water” provided, such as bottled water or filtered water. The non-purified water source can contain water meeting the WHO drinkable water standards provided in Guidelines for Drinking Water Quality, World Health Organization, Geneva, Switzerland, 4th edition, 2011. Alternatively, the water source 408 can be a source of purified water, meaning water that meets the applicable standards for use in peritoneal dialysis without additional purification. The system 400 pumps water from the water source 408 to the water purification module 410 to remove chemical contaminants in the fluid in preparation for creating dialysate. The water purification module 410 can be a sorbent cartridge containing anion and cation exchange resins and/or activated carbon. The system 400 can pump the fluid to a sterilization module 414 for sterilization of the peritoneal dialysate prior to infusion into the patient 450. The sterilization module 414 can include one or more of a first ultrafilter, a second ultrafilter, and a UV light source, or any combination thereof. The sterilization module 414 can be any component or set of components capable of sterilizing the peritoneal dialysate. The concentrate sources 412 can contain one or more solutes for generation of the peritoneal dialysate from purified water. The concentrates in the concentrate source 412 are utilized to create a peritoneal dialysis fluid that matches a dialysis prescription. A concentrate pump (not shown) in communication with the processor 422 or computing unit controls the movement of concentrates from the concentrate sources 412 into the peritoneal dialysate generation flow path 404. The concentrate sources 412 can include one or more sources of solutes for use in dialysis. One of skill in the art will understand that with a single concentrate source, solutes can be altered in the dialysate without changing the relative proportions of each solute. With multiple concentrate sources, each individual solute can be adjusted independently of all other solutes. Any number of concentrate sources and concentrate pumps can be used. A separate osmotic agent source and ion concentrate source can be used to adjust the osmotic agent concentration and other solute concentrations independently. Any solute usable in peritoneal dialysis can be included in the concentrate sources. The concentrate sources 412 can infuse each particular concentrate to provide an infused ion concentration that is lower than a prescribed amount for a particular patient 450. One desired outcome to be provide an concentration for a particular ion that is lower than a patient's pre-dialysis ion concentration. Additionally, if multiple ion sources are to be delivered by a concentrate source, the present system 400 can selectively dilute a desired ion while maintaining concentration levels for other ions. Hence, the present invention can avoid adjusting down every ion insofar as an added diluent may adversely affect concentrations of ions already in a normal range.

Fluid parameters can be derived from fluid sampled by one or more sensors 406 when removed from or introduced into the peritoneal cavity 452 of the patient 450. Fluid parameters can also be input into the system 400 as a parameter input 454 and/or received into the system 400 as prior history of the patient 456. A sensor 406 can be positioned in the peritoneal dialysate effluent line 440 of peritoneal dialysate generation flow path 404, or in both the peritoneal dialysate effluent line 440 and the infusion line (not shown) where separate effluent and infusion lines are used. A sensor 406 can be connected to the patient 450 or implanted in the patient 450. Fluid parameters can be derived using the one or more or more sensors 406. The sensors 406 can be separate sensors, a combined sensor positioned along separate peritoneal dialysate effluent lines and the infusion lines (not shown), or combined or separate sensors along a common peritoneal dialysate infusion line and effluent line 440. The sensors 406 can be placed at various locations along the peritoneal dialysate effluent line 440 and the peritoneal dialysate generation flow path 404, including within or between the cycler 416, the water source 408, the water purification module 410, the concentrate source 412, and the sterilization module 414, or between the cycler 416 and the peritoneal cavity 452 along the effluent line 440. The sensors 406 can be positioned to take measurements directly from the patient 450.

The one or more sensors 406 can include a fluid flow sensor to measure a net or given volume of fluid removed from a peritoneal cavity 452 of the patient 450. The sensor 406 can include a solute concentration sensor, such as a conductivity sensor or ion selective electrodes, to measure a solute concentration of the fluid removed from the patient 450. The sensor 406 can include a refractive index sensor to measure or other osmotic agent concentration in the fluid removed from the patient 450. The sensor 406 can include a pressure sensor to measure a pressure of fluid removed from a patient 450, or introduced into a patient 450. The sensor 406 can include a temperature sensor to measure a temperature of fluid removed from a patient 450, or introduced into a patient 450.

The computing device 420 can include the one or more processors 422, memory 424, and one or more input/output interfaces 426. The memory 424 can be any machine-readable storage medium in communication with the processor 422 and can store instructions that when executed by the processor 422 perform the methods. The input/output interfaces 426 can include an input interface to receive fluid parameter input 454, an input interface to receive prior history of the patient 456 of the patient 450, an input port to receive information from the one or more sensors 406, and an output interface to output alarms. The processor 422 can be in communication with the at least one sensor 406. As with all features of the present application, intervening components (such as the input/output interface 426) can be present between the processor 422 and the sensor 406. The computing device 420 can be a stand-alone device independent of the integrated cycler 416, or can be a part of the integrated cycler 416. The computing device 420 can be a remote device in network communication with the sensor 406, such as via the Internet.

Although illustrated as an integrated cycler 416, with a peritoneal dialysis generation flow path in FIG. 4, one of skill in the art will understand that a non-integrated peritoneal dialysis cycler can also perform the methods.

An alternative system for monitoring patient parameters for peritoneum and catheter health monitoring can include a peritoneal dialysate regeneration module, a pump, and an infusion line. The infusion line can be fluidly connected to the peritoneal dialysate generation flow path 404 downstream of the sterilization module 414. The peritoneal dialysate effluent line 440 can be fluidly connected to the peritoneal dialysate generation flow path 404 upstream of the peritoneal dialysate regeneration module. The peritoneal dialysate regeneration module can include a sorbent cartridge, an electrodialysis unit, one or more ultrafilters, or any other combination of components for removal of contaminants from the dialysate removed from the patient 450. The used peritoneal dialysate, after regeneration, can be pumped back into the peritoneal dialysate generation flow path 404 for reuse.

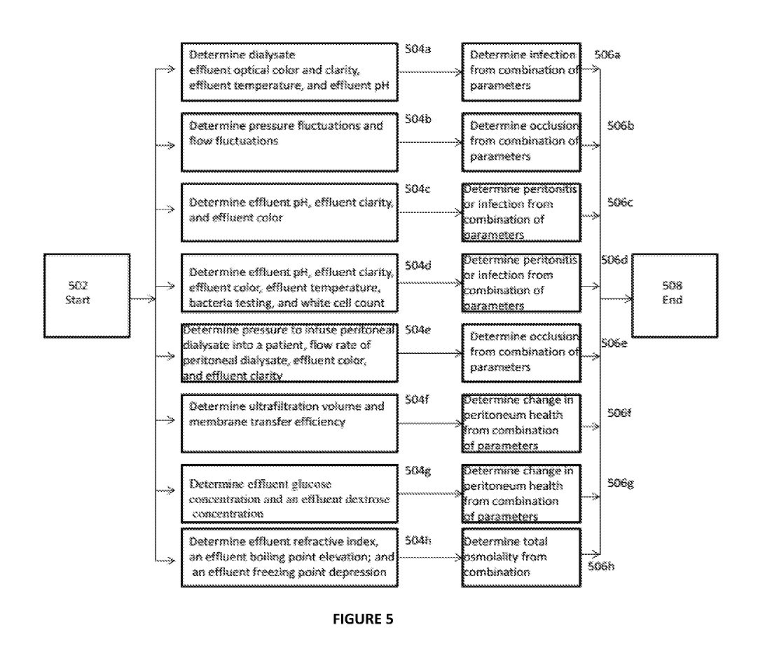

FIG. 5 is a flowchart of a computer implemented method for determining a change in peritoneum health of a patient based on one or more combinations of fluid parameters. The computer implemented method can be performed using a system configured to monitor fluid parameters during a peritoneal dialysis session, such as the system 400 of FIG. 4. Instructions for carrying out the method illustrated in FIG. 5 can be stored in a machine-readable storage medium. A processor in a dialysis machine can execute the instructions to perform the computer implemented method.

The method can begin in operation 502. A peritoneal dialysis session can already be underway. In operation 504, one or more combinations of fluid parameters can be received into the system during a peritoneal dialysis session. The fluid parameters can be stored in a database or any other machine-readable storage medium. The fluid parameters can be received into the system as input into an input/output interface of the system, or automatically received by the system from one or more sensors in communication with a processor of the system.

Multiple instances of operation 504 are depicted in FIG. 5. For example, in operation 504a, at least two of dialysate effluent optical color and clarity, effluent temperature, and effluent pH can be determined as described. In operation 504b, pressure fluctuations and flow fluctuations while infusing peritoneal dialysate into a patient can be determined. In operation 504c, at least two of effluent pH, effluent clarity, and effluent color can be determined. As another example, in operation 504d, at least two of effluent pH, effluent clarity, effluent color, effluent temperature, bacteria testing, and white cell count can be determined. In operation 504e, at least two of pressure to infuse peritoneal dialysate into a patient, flow rate of peritoneal dialysate, effluent color, and effluent clarity can be determined. In operation 504f, ultrafiltration volume and membrane transfer efficiency can be determined. In operation 504g, an effluent glucose concentration and an effluent dextrose concentration can be determined. In operation 504h, at least two of an effluent refractive index, an effluent boiling point elevation; and an effluent freezing point depression can be determined.

The method can proceed to operation 506. In operation 506, a change in peritoneum health can be determined based on the combinations of one or more fluid parameters. Multiple instances of operation 506 are depicted in FIG. 5. For example, in operation 506a, an infection can be determined using at least two of the parameters from operation 504a. In operation 506b, an occlusion in the catheter can be determined from the combination of parameters in operation 504b. In operation 506c, the presence of peritonitis or an infection can be determined from at least two of the parameters in operation 504c. In operation 506d, the presence of peritonitis or an infection can be determined from at least two of the parameters in operation 504d. In operation 506e, an occlusion in the catheter can be determined from a combination of at least two parameters in operation 504e. In operation 506f, a change in the peritoneum health of the patient can be determined from a combination of parameters in operation 504f. In operation 506g, a change in the peritoneum health of the patient can be determined from a combination of parameters in operation 504g. In operation 506h, the total osmolality can be determined from at least two parameters in operation 504h.

In operation 508, the method can end. If a worsening of peritoneum health or catheter status is determined in operation 506, an alert can be issued to the patient or health care professional indicating infection, catheter problems, or a failing peritoneum. The alert can indicate the need to intervene by altering the dialysate composition, changing the catheter, or transferring the patient to hemodialysis.

One skilled in the art will understand that various combinations and/or modifications and variations can be made in the systems and methods depending upon the specific needs for operation. Features illustrated or described as being part of an aspect of the invention may be used in the aspect of the invention, either alone or in combination.

1-12. (canceled)

13. A system, comprising:
a peritoneal dialysis cycler comprising an infusion line and an effluent line, or a combined effluent and infusion line;
at least one sensor positioned in the infusion line, the effluent line, or combinations thereof; and
a processor in communication with the at least one sensor; the processor performing the method of claim 1.

14. The system of claim 13, further comprising a detachable sampling reservoir fluidly connected to the effluent line or combined effluent and infusion line.

15. The system of claim 13, wherein the sensor includes at least one of a pressure sensor located in the infusion line or combined effluent and infusion line; a conductivity sensor positioned in the effluent line or combined effluent and infusion line; and/or a pH sensor positioned in the effluent line or combined effluent and infusion line.

16. A sensor suite for monitoring catheter and/or peritoneum health, wherein the sensor suite comprises at least one of:
a) a pressure sensor measuring a pressure infusing peritoneal dialysate into a patient;
b) an ultrafiltrate sensor measuring a membrane transfer efficiency across a peritoneal membrane, wherein the ultrafiltrate sensor measures at least one of: (i) toxin transfer; or (ii) protein leakage;
c) a fluid flow sensor; or
d) a dialysate effluent sensor measuring at least one of (i) effluent color; (ii) effluent clarity; or (iii) effluent temperature.

17. The sensor suite of claim 16, further comprising at least one of a pH sensor or a conductivity sensor.

18. The sensor suite of claim 16, wherein the sensor suite comprises at least two sensors.

19. The sensor suite of claim 16, wherein at least one sensor is positioned in a peritoneal dialysis cycler.

20. The sensor suite of claim 16, wherein the sensor suite includes at least the fluid flow sensor and the pressure sensor.

21. A sensor suite for dialysis performance and/or peritoneum health, comprising:
a group of sensors monitoring at least one of dialysis performance and/or peritoneum health condition selected from the group of inflammation, peritonitis, catheter obstruction for blockage of the catheter, peritoneal membrane health for transport status of the membrane, glucose concentration, total osmolality, and Kt/V for peritoneal dialysis, and/or hemodialysis, wherein the total osmolality is a sum of osmolalities of all solutes present in the peritoneal dialysis and wherein in Kt/V, K stands for dialyzer clearance of urea, t stands for dialysis time, and V stands for a volume of water the patient's body contains.

22. The sensor suite of claim 21, wherein the sensor suite is selected from the group of:
a) a pressure sensor measuring a pressure infusing a peritoneal dialysate into a patient;
b) an ultrafiltrate sensor measuring a membrane transfer efficiency across a peritoneal membrane, wherein the ultrafiltrate sensor measures at least one of: (i) toxin transfer; or (ii) protein leakage; and
c) a fluid flow sensor; or a dialysate effluent sensor measuring at least one of (i) effluent color; (ii) effluent clarity; or (iii) effluent temperature.

23. The sensor suite of claim 21, wherein dialysis performance Kt/V for peritoneal dialysis measures the conversion of urea to ammonia.

24. The sensor suite of claim 23, wherein dialysis performance Kt/V for peritoneal dialysis measures the conversion of urease to ammonia combined with the Kt/V measurement of residual kidney function.

25. The sensor suite of claim 21, further comprising one or more sensors monitoring dialysis performance via measuring catheter obstruction selected from the group of a flow rate sensor, pressure sensor, and an optical sensor.

26. The sensor suite of claim 21, further comprising one or more ultrafiltrate sensors monitoring peritoneum health measuring transport across the peritoneal membrane, Urea Reduction Ratio (URR) trending to determine changes in the URR over time, and urea trending to determine changes in urea over time in a peritoneal fluid.

27. The sensor suite of claim 21, further comprising one or more sensors monitoring peritoneum health selected from the group of a glucose sensor and a sensor measuring refractive index changes of dextrose in a peritoneal fluid.

28. The sensor suite of claim 21, further comprising one or more osmolality sensor monitoring peritoneum health selected from the group of a refractive index sensor, boiling point elevation, and freezing point depression.

29. The sensor suite of claim 21, further comprising one or more infection sensor monitoring peritoneum health selected from the group of a pH sensor, a color sensor, a clarity sensor, a temperature sensor, a bacterial concentration test, and a white cell concentration test.

30. The sensor suite of claim 21, further comprising one or more peritonitis sensor monitoring peritoneum health selected from the group of a pH sensor, a color sensor, a clarity sensor, a temperature sensor, a bacterial concentration test, and a white cell concentration test.
